(19) United States
(12) Patent Application Publication
(10) Pub. No.: US 2024/0389960 A1
(43) Pub. Date: Nov. 28, 2024

(54) COMPUTED TOMOGRAPHY SYSTEM AND METHOD FOR OPERATING A COMPUTED TOMOGRAPHY SYSTEM

(71) Applicant: Siemens Healthineers AG, Forchheim (DE)

(72) Inventors: Thomas FLOHR, Uehlfeld (DE); Karl STIERSTORFER, Erlangen (DE)

(73) Assignee: Siemens Healthineers AG, Forchheim (DE)

(21) Appl. No.: 18/646,882

(22) Filed: Apr. 26, 2024

(30) Foreign Application Priority Data
May 26, 2023 (EP) 23175738.6

(51) Int. Cl. A61B 6/00; A61B 6/40; A61B 6/42

(52) U.S. Cl. CPC A61B 6/482; A61B 6/4014; A61B 6/4035; A61B 6/4241

(57) ABSTRACT

A computed tomography system comprising an annular x-ray source having a plurality of partial x-ray sources around an examination region and a detector annulus having a plurality of x-ray detectors around an examination region. The computed tomography system is configured to activate the partial x-ray sources individually during a computed tomography scan using a static x-ray source and a static detector annulus to generate absorption profiles of an object in the examination region from a plurality of directions. The computed tomography system is configured to at least one of generate at least a first x-ray spectrum using a first group of the partial x-ray sources and a second x-ray spectrum, distinguishable from the first, using a second group of the partial x-ray sources during the computed tomography scan, or is configured to record an x-ray signal in a spectrally distinguishable manner using the x-ray detectors.

### CROSS-REFERENCE TO RELATED APPLICATION(S)

The present application claims priority under 35 U.S.C. § 119 to European Patent Application No. 23175738.6, filed May 26, 2023, the entire contents of which is incorporated herein by reference.

### FIELD

One or more example embodiments of the present invention relates to a computed tomography system and a method for operating a computed tomography system.

Independent of the grammatical term usage, individuals with male, female or other gender identities are included within the term.

### RELATED ART

In the context of examinations based on computed tomography (CT), it is desirable to be able to provide further functional information in addition to a purely anatomical depiction. Via simultaneous or very slightly time-staggered recording of CT data relating to an object or subject under examination, in particular a patient, it is possible to obtain for example information relating to a chemical composition of the examined regions using various average x-ray energies. Alternatively, it is thereby also possible separately to represent and quantify specific substances or materials, for example iodine-containing contrast media, in material-specific images. A known concept is the recording of two CT datasets using different average energies, this being referred to as “dual-energy CT”.

The use of third-generation CT systems is customary in the prior art. In these CT systems, an x-ray source and an opposite x-ray detector together rotate about a subject under examination. So-called dual-source CT devices are available for the purpose of implementing the dual-energy CT concept, in which two third-generation measuring systems rotate about the subject, said measuring systems being offset by 90°. The two measuring systems can be operated simultaneously using various acceleration voltages, for example, so that two CT datasets can be recorded simultaneously using different average energies.

### SUMMARY

The rotation of two CT systems and the resulting forces require a correspondingly robust construction which therefore also occupies additional space. The non-trivial mechanical demands can also result in increased servicing expense.

The object of the present invention is to find an alternative for the recording of various energies or spectral data using a CT system, which alternative can preferably be implemented using a mechanically less expensive and/or potentially space-saving construction.

This object is achieved by a computed tomography system as claimed in claim 1 and a method for operating a computed tomography system as claimed in claim 15.

### BRIEF DESCRIPTION OF THE DRAWINGS

Embodiments are described in the following with reference to the appended figures, in which:

FIG. 1 shows a computed tomography system according to an embodiment of the invention,

FIG. 2 shows an annular x-ray source and a detector annulus of a computed tomography system according to an embodiment of the invention,

FIG. 3 shows an annular x-ray source and a detector annulus of a computed tomography system according to a further embodiment of the invention,

FIG. 4 shows an annular x-ray source and a detector annulus of a computed tomography system according to a further embodiment of the invention,

FIG. 5 shows an annular x-ray source and a detector annulus of a computed tomography system according to a further embodiment of the invention,

FIG. 6 shows an annular x-ray source and a detector annulus of a computed tomography system according to a further embodiment of the invention,

FIG. 7 shows an annular x-ray source and a detector annulus of a computed tomography system according to a further embodiment of the invention, and

FIG. 8 shows a flow diagram of a method for operating a computed tomography system according to an embodiment of the invention.

### DETAILED DESCRIPTION

According to one or more example embodiments of the present invention, provision is made for a computed tomography system. The computed tomography system comprises an annular x-ray source having a plurality of partial x-ray sources which are arranged around an examination region in the form of a circle or part-circle, and a detector annulus having a plurality of x-ray detectors which are arranged around an examination region in the form of a circle or part-circle, wherein the computed tomography system is configured to activate at least some of the partial x-ray sources individually one after the other during a computed tomography scan using a static x-ray source and a static detector annulus and thereby to generate absorption profiles of an object in the examination region from a plurality of directions using the x-ray detectors, wherein the computed tomography system is configured to generate at least a first x-ray spectrum using a first group of the partial x-ray sources and a second x-ray spectrum, distinguishable from the first, using a second group of the partial x-ray sources during the computed tomography scan, and/or is configured to record an x-ray signal in a spectrally distinguishable manner using the x-ray detectors.

The examination region can be embodied in particular such that a subject or object under examination, in particular a patient, can be arranged or situated at least partially therein. The partial x-ray sources in each case can be in particular individual x-ray sources which are arranged according to the annulus of the annular x-ray sources. Provision can optionally be made for partial x-ray sources within one of the groups or both of the groups to be further divided into subgroups. The subgroups can comprise a plurality of partial x-ray sources in each case. At least one of the subgroups can optionally also contain only a single partial x-ray source. The x-ray sources can preferably be arranged at different angular positions of the annular x-ray source. In particular, the x-ray sources can be uniformly distributed around the annulus of the x-ray source. This means that x-ray projections through the examination region can advantageously be generated from various angular positions. In particular, this allows a computed tomography image to be reconstructed in the same way as an image from a computed tomography system with a rotating scanner. The annular x-ray source can comprise a basic structure on or along which the partial x-ray sources are arranged. The annular x-ray source can comprise for example a plurality of cathodes, in particular angularly offset cathodes, and be configured to generate a high voltage between the cathodes and an anode, so that electrons are emitted from the respective cathode, guided to the anode by the high voltage, form a focal spot there and generate an x-ray fan beam. This x-ray fan beam can preferably be directed at the examination region and captured by the x-ray detectors. The cathodes can be for example field emission cathodes, in particular nanotubes. The annular x-ray source can form a complete annulus or a partial annulus. Accordingly, the partial x-ray sources can be arranged in the form of a part-circle in the case of a partial annulus or in the form of a circle in the case of a complete annulus. The x-ray detectors can be or comprise detector elements in each case. The x-ray detectors can preferably be arranged opposite the partial x-ray sources in each case. The detector annulus can be a complete annulus or a partial annulus. Accordingly, the x-ray detectors can be arranged in the form of a part-circle in the case of a partial annulus or in the form of a circle in the case of a complete annulus. According to an embodiment, the annular x-ray source and the detector annulus are in each case partial annuli which are arranged opposite each other so that the examination region is situated between them. Partial annuli can be advantageous if irradiation is not required from all directions, in particular in order to save costs or materials, or allow the system to be embodied with a simpler structure. According to an embodiment, the annular x-ray source and the detector annulus are distributed over a complete annulus in each case. In the case of a complete annulus, it is advantageously possible to irradiate the subject or object from all directions. The annular x-ray source and the detector annulus can be combined to form a single annulus. For example, the annular x-ray source and the detector annulus can each be partial annuli which together form a shared complete annulus. Alternatively, both the x-ray source and the detector annulus can each extend over a complete annulus. For example, the partial x-ray sources can be arranged in each case alternately with the x-ray detectors serially in a circumferential direction and/or offset relative to each other in a radial direction. For example, the partial x-ray detectors can be arranged at a first distance from a center of the shared annulus and the x-ray detectors at a second distance from the center. Alternatively or additionally, the annular x-ray source and the detector annulus can be offset relative to each other in a longitudinal direction parallel to the normal of the circular area of the annulus. It is also conceivable for either of the detector annulus and the annular x-ray source to be designed as a partial annulus and the other as a complete annulus.

According to a first advantageous variant, it is intended that both x-ray source and detector annulus are held static during a computed tomography scan, meaning in particular that they are not rotated about the examination region. Therefore static can be understood to mean in particular that no physical rotation of the scanner about the examination region takes place during a scan. The avoidance of rotation of the scanner in the CT system means that it is advantageously possible to use a less robust construction due to the absence of rotational forces and due to the consequently simpler requirements resulting from the static and immobile embodiment. It may also be possible to reduce the servicing expense because there may be less wear without rotation. The rotation can be omitted because certain of the partial x-ray sources can be activated individually one after the other and therefore absorption profiles of the object in the examination region can be generated from a plurality of directions via the x-ray detectors arranged in the form of a (part-) circle. For example, the partial x-ray sources can be activated in a circumferential direction according to their sequence. It is however also conceivable to specify a different activation sequence. The inventive system also allows spectral data to be recorded. This can be achieved via generating at least two different x-ray spectra using the at least two groups of partial x-ray sources. In this case, distinguishable is understood to mean in particular that it is possible, on the basis of the recording by the x-ray detectors of the received x-rays, to distinguish between the two x-ray spectra. Provision can be made such that an energy distribution of the first x-ray spectrum differs significantly from an energy distribution of the second x-ray spectrum. For example, the first x-ray spectrum can have a higher average energy than the second x-ray spectrum or vice versa. Provision can be made such that a major part of the energy range of the first x-ray spectrum does not overlap a major part of the energy range of the second x-ray spectrum. A major part can be more than 50 percent, preferably more than 60 percent, most preferably more than 75 percent, of the respective x-ray spectrum. Provision can be made such that the first group of partial x-ray sources is activated or operated initially, followed by the second group of partial x-ray sources. Provision can be made such that more than two groups of partial x-ray sources are activated one after the other.

Alternatively, according to a second variant, the recording of spectral data can be made possible by enabling the x-ray detectors to record an x-ray signal in a spectrally distinguishable manner. For example, provision can be made such that all partial x-ray sources generate an essentially identical spectrum and that this spectrum can automatically be divided into two partial spectra, for example one above and one below a defined energy threshold value. It is thus advantageously possible for a spectral capture to be made possible by the partial x-ray sources.

It is however also conceivable to combine these two variants into a third variant. Accordingly, provision can be made such that for example one of the group of partial x-ray sources records the first x-ray spectrum and the second group of partial x-ray sources records the second x-ray spectrum, while the x-ray detectors record the received x-ray signals in a spectrally distinguishable manner. It is thereby possible for example to improve the ability to distinguish between the at least two x-ray spectra, or spectral separation. However, provision can also be made to effect a division into an even greater number of distinguishable spectra via this combination. For example, the system can be configured such that the first x-ray spectrum and the second x-ray spectrum are each divided again by the x-ray detectors into two sub-spectra. This means that it is advantageously possible to obtain a larger number of different spectra, in particular with different average energies in each case. For example, the x-ray detectors can be configured to distinguish between four different energy ranges, and provision can be made for two groups of partial x-ray sources and therefore two x-ray spectra that are generated by the annular x-ray source. This example advantageously allows computed tomography data with eight (4 times 2) different average energies.

According to an embodiment, the computed tomography system is configured to generate the first x-ray spectrum by applying a first acceleration voltage at the partial x-ray sources of the first group of the partial x-ray sources, and the second x-ray spectrum by applying a second acceleration voltage at the partial x-ray sources of the second group of the partial x-ray sources. The first or second acceleration voltage respectively is applied in particular between a cathode of the respective partial x-ray source and an associated anode. It has been shown that using two (or optionally more) acceleration voltages, good spectral distinguishability can be achieved using this embodiment. For example, the first acceleration voltage can lie in the range from 50 kV to 100 kV, preferably in the range from 70 kV to 90 kV. For example, the second acceleration voltage can lie in the range from 100 kV to 200 kV, preferably in the range from 120 kV to 170 kV, most preferably in the range from 130 kV to 150 kV. A specific example can be a first acceleration voltage of 80 kV and a second acceleration voltage of 140 kV. It has been shown that particularly good results can be obtained using such values, in particular because the spectra can be easily distinguishable and at the same time lie in a range which is favorable for the imaging. For example, provision can be made such that partial x-ray sources are activated one after the other, wherein when switching from one partial x-ray source (or cathode) or subgroup (or cathode group) to the next, the acceleration voltage is likewise switched. It is thereby possible (with the aid of the x-ray detectors) to record in particular temporally consecutive x-ray projections with a different x-ray spectrum or different average x-ray energy. Alternatively, it is also conceivable for a plurality of consecutively activated partial x-ray sources to be activated using the same acceleration voltage in each case, and for the acceleration voltage to be changed after switching a predetermined number of times. In other words, the system can be configured to consecutively activate partial x-ray sources of the same group (for example only the first group) and then to consecutively activate partial x-ray sources of the other group (for example only the second group). In the case of two groups, the computed tomography system can be a dual-energy system in particular.

According to an embodiment, the annular x-ray source comprises a first voltage connection for generating an acceleration voltage for the first group of the partial x-ray sources and a second voltage connection for generating an acceleration voltage for the second group of the partial x-ray sources. The x-ray source can optionally comprise one or more further voltage connections for one or more further groups. The voltage connections can preferably be circular or part-circular in design, corresponding to the annular form of the x-ray source. For example, the first voltage connection can be embodied to generate the first acceleration voltage as described herein and the second voltage connection can be embodied to generate the second acceleration voltage as described herein. According to an embodiment, the first and the second voltage connection are configured to supply an acceleration voltage to the partial x-ray sources individually or in subgroups.

According to an embodiment, the computed tomography system is configured to activate at least one of the partial x-ray sources of the first group and at least one of the partial x-ray sources of the second group alternately in each case. The system can be configured to activate subgroups of the first group and subgroups of the second group alternately one after the other in each case. For example, the system can be configured to activate a partial x-ray source of the first group and a partial x-ray source of the second group alternately in each case, in particular in such a way that all partial x-ray sources are activated one after the other. For example, the first group can comprise a plurality of subgroups of partial x-ray sources. At least some of the subgroups preferably consist of a plurality of partial x-ray sources in each case. For example, the system can be configured to activate a subgroup of the first group and a subgroup of the second group alternately in each case, in particular in such a way that all subgroups are activated one after the other. At least one of the subgroups can optionally consist of an individual partial x-ray source. The system can be configured to activate alternately in each case a partial x-ray source or a subgroup of the first group using the first acceleration voltage and a partial x-ray source or a subgroup of the second group using the second acceleration voltage. The activation is effected in particular by applying the first or second acceleration voltage respectively.

According to an embodiment, the partial x-ray sources are arranged on the annular x-ray source in such a way that a partial x-ray source of the first group and a partial x-ray source of the second group are arranged alternately in each case in a circumferential direction. It can advantageously be possible thus to achieve a particularly good spatial distribution of the at least two different x-ray spectra. The computed tomography system is optionally also configured to activate the partial x-ray sources in the sequence of their arrangement on the circular circumference. It can advantageously be possible thus to achieve a particularly good temporal distribution of the at least two different x-ray spectra. It is thereby possible if necessary to reduce errors in a spectral analysis caused by movement of the subject or object.

According to an embodiment, the computed tomography system is embodied to generate the second x-ray spectrum by adapting the x-radiation which is generated by the second group of partial x-ray sources, using x-ray filters. An identical type of filters is preferably provided for all partial x-ray sources of the second group. An identical type in this case refers in particular to the filter properties in the x-ray spectrum. The x-ray filters are preferably embodied to filter out or at least attenuate certain energies, in particular an energy range, from the spectrum. The x-ray filters can preferably be embodied to filter out x-ray energies which lie below an energy threshold. In other words, the x-ray filters can be embodied to harden the x-ray spectrum. By providing the x-ray filters, it is consequently possible to generate two distinguishable x-ray spectra. This variant represents a particularly favorable alternative, in particular because it does not necessarily require the provision of a second voltage connection. Provision can advantageously be made for combining this embodiment with the embodiment that uses a first and a second acceleration voltage. It has been shown that the spectral separation, which can already be achieved very effectively via two acceleration voltages, can be further improved by the additional use of the x-ray filters. The acceleration voltages typically generate an energy spectrum. A maximum energy is typically defined by the acceleration voltage, while an energy range in which a number of photons is not negligible can nonetheless be considerably lower. For example, the generated x-ray spectrum can have relevant parts with an acceleration voltage from 140 kV to 30 kV in some circumstances. The width of the x-ray spectrum that is actually measured can also depend on a sensitivity of the x-ray detectors. An x-ray spectrum can be relatively wide accordingly. An overlap of the two generated x-ray spectra can therefore occur in practice. By providing the x-ray filters, in particular filters in front of the second group with a higher acceleration voltage, the respective x-ray spectrum can be limited accordingly in order to prevent or at least reduce any overlap of the two spectra. Using this combination, a filter in front of the partial x-ray sources of the second group can be particularly advantageous if it filters out x-ray energies which lie below an energy threshold. The energy threshold of the x-ray filters can preferably lie between the energy corresponding to the first acceleration voltage and the energy corresponding to the second acceleration voltage. In the case of acceleration voltages of 80 kV and 140 kV, the energy threshold can therefore preferably lie in the range between 80 keV and 140 keV. The x-ray filters can be tin filters, for example. By virtue of their filter properties, tin filters can be particularly suitable as the filter for the second group of partial x-ray sources. Corresponding to the arrangement of the partial x-ray sources of the first group and the second group, the x-ray filters can be arranged in such a way that for example every second partial x-ray source in a circumferential direction is fitted with an x-ray filter. In an alternative arrangement of the partial x-ray sources of the first group and the second group, for example a plurality of adjacent partial x-ray sources can be provided with an x-ray filter. The x-ray filters can optionally be embodied in such a way that a first part of the emitted x-radiation of an x-ray source is filtered by a first filter part and a second part is filtered by a second filter part or is not filtered at all, wherein the spatial diffusion of the first part and the second part differs in such a way that the first part is captured by different x-ray detectors or detector elements than the second part. The x-ray detectors can be for example multirow detectors with row-by-row detector elements. For example, half of a multirow detector, i.e. in particular half of the detector elements, can capture an x-ray spectrum having a higher average energy than the other half, i.e. in particular the other half of the detector elements of the detector. This allows a further spectral distribution.

According to an embodiment, the computed tomography system is embodied to generate the first x-ray spectrum by likewise adapting the x-radiation which is generated by the first group of partial x-ray sources, using further x-ray filters. The further x-ray filters can preferably differ from the x-ray filters of the second group in respect of their filter properties. The further x-ray filters can preferably be embodied to shift an average energy of the x-ray spectrum to lower values. For example, the further x-ray filters can be gold filters. The further x-ray filters (in addition to the x-ray filters in front of the partial x-ray sources of the second group) can further improve the spectral distinguishability. This further embodiment can likewise be advantageously combined with the embodiment that uses a first and a second acceleration voltage, in particular to further improve spectral distinguishability.

According to an embodiment, the annular x-ray source comprises filter slots, at least in front of the second group of partial x-ray sources, for the manual or automatic insertion of x-ray filters. Alternatively or additionally the annular x-ray source can comprise x-ray filters at least in front of the second group of partial x-ray sources. The filter slots are preferably embodied in such a way that the x-ray filters, when inserted in the filter slots, are situated in front of the corresponding partial x-ray sources. The filter slots can allow a simple exchange of the filters. This allows the generation of different x-ray spectra, for example. For each of the filter slots, the computed tomography system can comprise a loading mechanism for the x-ray filters, said loading mechanism being automatic in particular. For example, the system can be embodied to unload the filters from their position in front of the partial x-ray sources in such a way that they can be removed or exchanged by a user. The system can comprise a filter storage region in which the filters can be stored when not situated in front of the partial x-ray sources. The loading mechanism can be embodied to move the x-ray filters from the filter slots into the filter storage region and back.

According to an embodiment, the computed tomography system is configured to generate, during the computed tomography scan, one or more further x-ray spectra using one or more further groups of the partial x-ray sources, including at least a third group. In other words, the system can be configured to activate more than two groups using a respective acceleration voltage and a respective x-ray spectrum in each case. For example, the system can be configured to additionally generate at least a third x-ray spectrum by applying a third acceleration voltage to a third group of the partial x-ray sources. Using this embodiment, it can advantageously be possible to spectrally resolve more than two energies.

According to an embodiment, the detector annulus comprises photon-counting detectors which are configured to distinguish between at least two energy thresholds. This means that photon-counting detectors can advantageously be used to allow the generation of spectral data. The detectors can preferably be embodied to generate two datasets from captured x-radiation in each case, a first dataset comprising energies above a first threshold value and a second dataset comprising energies above a second threshold value which is greater than the first threshold value.

According to an embodiment, the x-ray detectors are provided as detector pairs, one of which absorbs essentially low-energy x-radiation while the other absorbs essentially high-energy x-radiation. In particular, the x-ray detectors can be dual-layer detectors. Dual-layer detectors consist in particular of two detectors arranged one above the other in a beam direction, the upper detector, which is closer to the x-ray source, primarily absorbing low-energy x-ray quanta and the lower absorbing the remaining higher-energy x-ray quanta. The photon-counting detectors or the detector pairs can be combined with the embodiment according to which a first x-ray spectrum and a second x-ray spectrum are generated by two groups of partial x-ray sources, in particular with the specific embodiment which uses at least two different acceleration voltages. The system can be configured to dynamically adjust the threshold values of the detectors and to synchronize said threshold values with the application of the acceleration voltages. This combination can further improve spectral separation or distinguishability. Alternatively, it can be possible to generate a larger amount of spectrally distinguishable x-ray data. For example, the combination of photon-counting detectors using four energy thresholds with voltage switching between two different acceleration voltages, corresponding to the two groups, can result in computed tomography data having eight different average energies.

One or more example embodiments of the present invention is a method for operating a computed tomography system which has a plurality of partial x-ray sources and a plurality of x-ray detectors, each being operated in a positionally fixed manner and surrounding an examination region, comprising the following steps:
(a) activating a first partial x-ray source or a first subgroup of partial x-ray sources in order to generate x-radiation that is directed at the examination region and has a first x-ray spectrum;
(b) activating a second partial x-ray source or a second subgroup of partial x-ray sources in order to generate x-radiation that is directed at the examination region and has a second x-ray spectrum which is different than the first x-ray spectrum;
(c) detecting the x-ray beams of the first and the second x-ray spectra which pass through the examination region via the x-ray detectors.

The steps a and b can preferably be performed in a time-staggered manner. The steps a, b and c can most preferably be repeated, different partial x-ray sources or subgroups being activated in each case in the steps a and b. In particular, partial x-ray sources or subgroups can each be activated alternately as per step a and step b in a circumferential direction. The partial x-ray sources and the x-ray detectors are preferably distributed in a circular manner around the examination region, in particular in such a way that respectively individual x-ray detectors are arranged opposite respectively individual partial x-ray sources. All of the advantages and features of the system can be transferred analogously to the method and vice versa.

All of the embodiments described herein can be combined with each other unless explicitly specified otherwise.

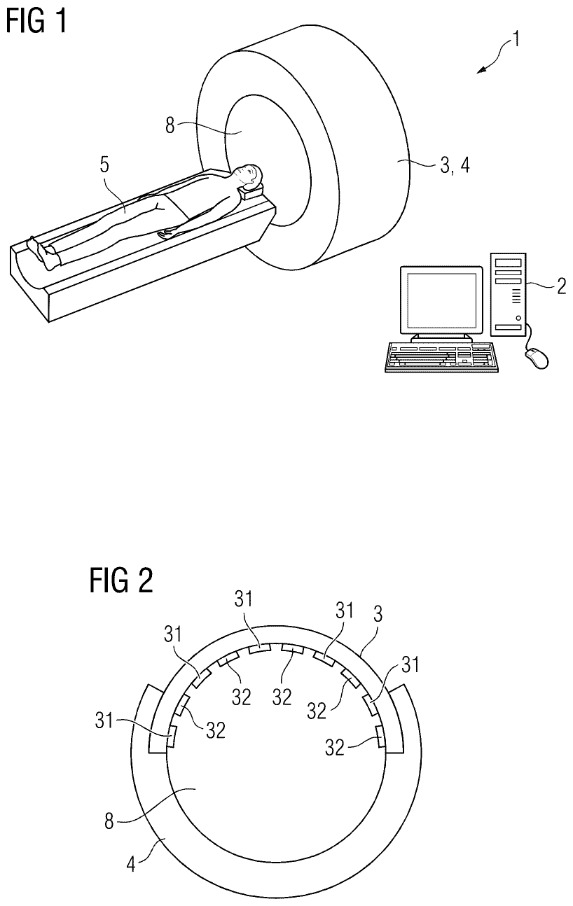

FIG. 1 shows a computed tomography system 1 according to an embodiment of the invention. The system 1 comprises an examination region 8 around which an x-ray source 3 and a detector annulus 4 are arranged in an annular manner. For the purpose of a computed tomography scan, a subject 5, in particular a patient, can be moved into the examination region 8. The computed tomography system 1 comprises a control unit 2, for example a computer, which is configured to control the system 1. The x-ray source 3 and the detector annulus 4 can be embodied as shown in the following FIGS. 2 to 5, for example.

FIG. 2 shows an annular x-ray source 3 and a detector annulus 4 of a computed tomography system 1 according to an embodiment of the invention. The annular x-ray source 3 and the detector annulus 4 are embodied in each case as a partial annulus and arranged around an examination region 8. In this case, x-ray detectors 41 (not shown here) which are arranged in the form of a part-circle along the detector annulus 4 are positioned opposite partial x-ray sources 31, 32 of the annular x-ray source 3 which arranged in the form of a part-circle. The system 1 is configured to generate, during a computed tomography scan, a first x-ray spectrum using a first group of the partial x-ray sources 31 and a second x-ray spectrum, distinguishable from the first, using a second group of the partial x-ray sources 32. The computed tomography system 1 can optionally be configured to activate alternately at least one of the partial x-ray sources 31 of the first group and at least one of the partial x-ray sources 32 of the second group. In the arrangement shown here of the partial x-ray sources 31, 32, in which partial x-ray sources 31 of the first group and partial x-ray sources 32 of the second group are provided in each case alternately in a circumferential direction, the system 1 can be configured thereby in particular to activate the partial x-ray sources 31, 32 in the sequence of their arrangement on the circular circumference.

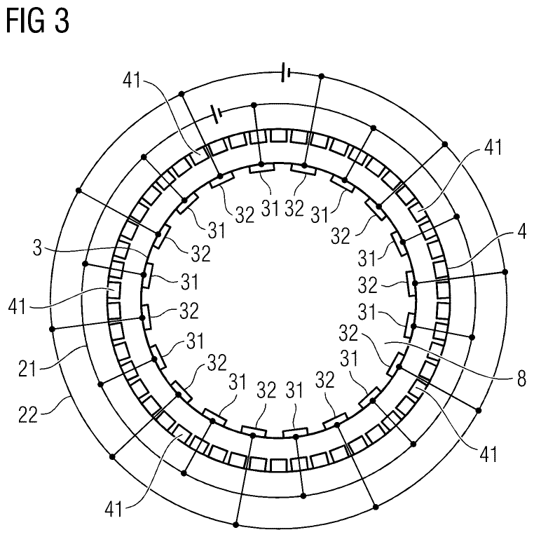

FIG. 3 shows an annular x-ray source 3 and a detector annulus 4 of a computed tomography system 1 according to a further embodiment of the invention. The annular x-ray source 3 and the detector annulus 4 are distributed over a complete annulus in each case. The annular x-ray source 3 comprises a plurality of partial x-ray sources 31, 32 which are arranged in the form of a circle around an examination region 8. The detector annulus 4 comprises a plurality of x-ray detectors 41 which are arranged around the examination region 8 in the form of a circle and opposite the partial x-ray sources 31, 32 in each case. In this and in the following embodiments, the partial x-ray sources 31, 32 and the x-ray detectors 42 are preferably offset relative to each other in an axial direction, i.e. in a direction extending perpendicularly into the plane of the image. The system 1 is configured to generate, during a computed tomography scan, a first x-ray spectrum using a first group of the partial x-ray sources 31 and a second x-ray spectrum, distinguishable from the first, using a second group of the partial x-ray sources 32. In particular, the first and the second x-ray spectra can have different average energies. In particular, there is no rotation of the x-ray source 3 or the detector annulus 4 during the recording. The recording is preferably carried out by generating absorption profiles of a subject 5 or object in the examination region 8 from a plurality of directions as a result of activating individual partial x-ray sources 31, 32, these being arranged at different angles, in combination with the x-ray detectors 41. To this end, the annular x-ray source 3 comprises a first voltage connection 21 for generating an acceleration voltage for the first group of the partial x-ray sources 31 and a second voltage connection 22 for generating an acceleration voltage for the second group of the partial x-ray sources 32. The first x-ray spectrum is generated by applying the first acceleration voltage at the partial x-ray sources 31 of the first group, and the second x-ray spectrum is generated by applying a second acceleration voltage at the partial x-ray sources 32 of the second group. The partial x-ray sources 31, 32 can be activated in particular in the sequence of their arrangement on the circular circumference of the annular x-ray source 3. For example, via switching rapidly back and forth between the two acceleration voltages, it is possible to record consecutive CT projections using the first x-ray spectrum and the second x-ray spectrum alternately in each case. Alternatively, if the recording of the CT data relates to an immobile subject 5 or object, i.e. with no time-related changes of a contrast medium concentration for example, the switching back and forth between the two acceleration voltages can optionally also take place more slowly. For example, a plurality of projections can be recorded using one acceleration voltage followed by a plurality of projections using the other acceleration voltage. The system 1 can optionally be configured also to record an x-ray signal in a spectrally distinguishable manner using the x-ray detectors 41 during the computed tomography scan. The x-ray detectors 41 can be photon-counting detectors, for example, which are configured to distinguish between at least two energy thresholds. This combination can further improve the spectral separation. Alternatively or additionally, this combination also allows the number of distinguishable x-ray spectra to be increased, for example to a total of four, using two energy thresholds of the x-ray detectors 41.

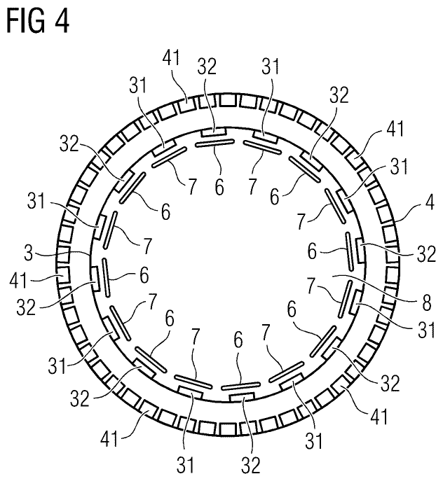

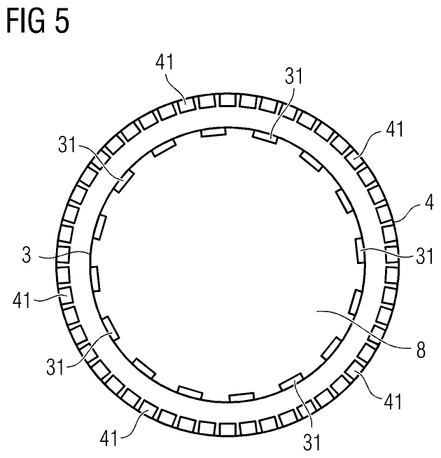

FIG. 4 shows an annular x-ray source 3 and a detector annulus 4 of a computed tomography system 1 according to a further embodiment of the invention. The annular x-ray source 3 and the detector annulus 4 are distributed over a complete annulus in each case. The annular x-ray source 3 comprises a plurality of partial x-ray sources 31, 32 which are arranged in the form of a circle around an examination region 8. The detector annulus 4 comprises a plurality of x-ray detectors 41 which are arranged around the examination region 8 in the form of a circle and opposite the partial x-ray sources 31, 32 in each case. The computed tomography system 1 is embodied to generate the second x-ray spectrum and the first x-ray spectrum differently, specifically by using the x-ray filters 6 to adapt the x-radiation that is generated by the second group of partial x-ray sources 32. This means that in particular the acceleration voltage of all partial x-ray sources 31, 32 can be identical and the distinguishability can be brought about solely by the x-ray filters 6. It is however optionally also possible in each case to provide further x-ray filters 7 in front of the first group of partial x-ray sources 31, which further x-ray filters 7 differ from the x-ray filters 6 of the second group in respect of their filter properties. It is thereby possible to further improve the ability to distinguish between the two x-ray spectra. Filter slots can be provided for the manual or automatic insertion of the x-ray filters 6, 7. These can normally be provided in all embodiments in which x-ray filters 6, 7 are used.

FIG. 5 shows an annular x-ray source 3 and a detector annulus 4 of a computed tomography system 1 according to a further embodiment of the invention. The annular x-ray source 3 and the detector annulus 4 are distributed over a complete annulus in each case. The annular x-ray source 3 comprises a plurality of partial x-ray sources 31, 32 which are arranged in the form of a circle around an examination region 8. The detector annulus 4 comprises a plurality of x-ray detectors 41 which are arranged around the examination region 8 in the form of a circle and opposite the partial x-ray sources 31, 32 in each case. The computed tomography system 1 is configured to record, during a computed tomography scan, an x-ray signal in a spectrally distinguishable manner using the x-ray detectors 41, and thus to generate spectral data. To this end, the x-ray detectors 41 can be for example photon-counting detectors which are configured to distinguish between at least two energy thresholds and thus to record two distinguishable x-ray spectra.

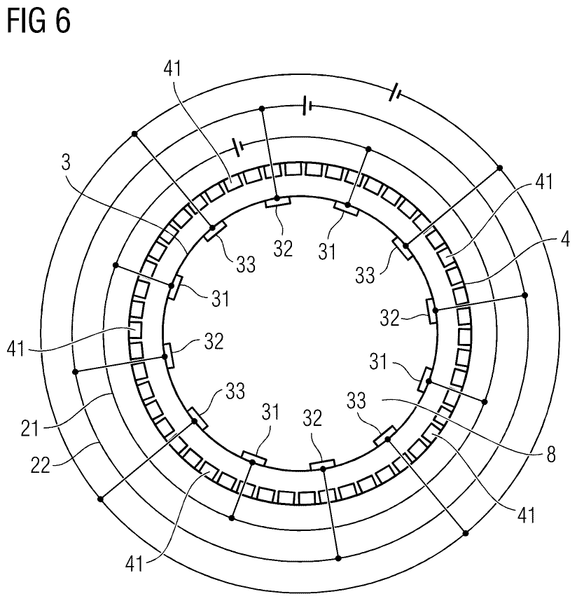

FIG. 6 shows an annular x-ray source 3 and a detector annulus 4 of a computed tomography system 1 according to a further embodiment of the invention. This embodiment can correspond essentially to that shown in FIG. 3, but nonetheless differs in that the computed tomography system 1 is configured to generate, during the computed tomography scan, a further x-ray spectrum using a third group of partial x-ray sources 33. It is thereby possible to generate spectral data having three different energy levels.

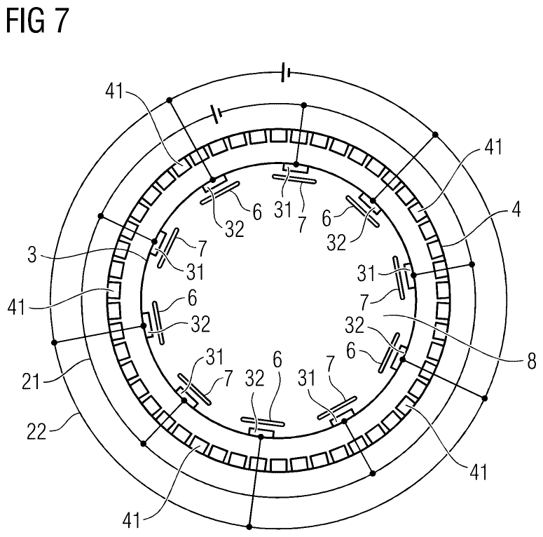

FIG. 7 shows an annular x-ray source 3 and a detector annulus 4 of a computed tomography system 1 according to a further embodiment of the invention. This embodiment can correspond essentially to that shown in FIG. 3, but is however developed in that the x-radiation which is generated by the second group of partial x-ray sources 32 is adapted using x-ray filters 6. It is thereby possible to further improve the ability to distinguish between the two generated x-ray spectra. It is however optionally also possible in each case to provide further x-ray filters 7 in front of the first group of partial x-ray sources 31, said further x-ray filters 7 differing from the x-ray filters 6 of the second group in respect of their filter properties. It is thereby possible to further improve the ability to distinguish between the two x-ray spectra.

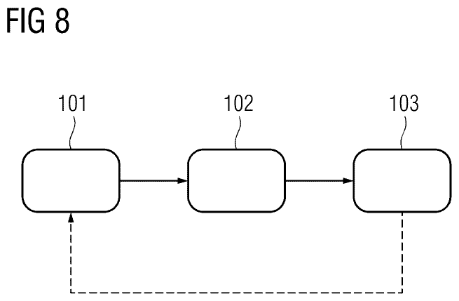

FIG. 8 shows a flow diagram of a method for operating a computed tomography system 1 according to an embodiment of the invention. In this case, a plurality of partial x-ray sources 31, 32 and a plurality of x-ray detectors 41, which surround an examination region, are operated in a positionally fixed manner. In a first step 101, a first partial x-ray source 31 or a first subgroup of partial x-ray sources 31 is activated in order to generate x-radiation that is directed at the examination region 8 and has a first x-ray spectrum. In a step 102 following thereupon, a second partial x-ray source 32 or a second subgroup of partial x-ray sources 32 is activated in order to generate x-radiation that is directed at the examination region 8 and has a second x-ray spectrum which is different than the first x-ray spectrum. In a further step 103, the x-ray beams of the first and the second x-ray spectra which pass through the examination region 8 are detected via the x-ray detectors 41. These steps 101-103 are preferably repeated multiple times for different partial x-ray sources 31, 32, in particular until all partial x-ray sources have been activated.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 or degrees at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to being “directly” on, connected, engaged, interfaced, d to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, non-transitory, the tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple elements or processing processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computed tomography system comprising:
an annular x-ray source having a plurality of partial x-ray sources around an examination region in the form of a first circle or a first part-circle; and
a detector annulus having a plurality of x-ray detectors around the examination region in the form of a second circle or a second part-circle, wherein the computed tomography system is configured to,
activate at least some of the partial x-ray sources individually during a computed tomography scan using a static x-ray source and a static detector annulus, and
generate absorption profiles of an object in the examination region from a plurality of directions using the x-ray detectors,
wherein the computed tomography system is further configured to at least one of,
generate at least a first x-ray spectrum using a first group of the partial x-ray sources and a second x-ray spectrum, distinguishable from the first x-ray spectrum, using a second group of the partial x-ray sources during the computed tomography scan, or
record an x-ray signal in a spectrally distinguishable manner using the x-ray detectors.

2. The computed tomography system of claim 1, wherein the computed tomography system is configured to generate the first x-ray spectrum by applying a first acceleration voltage at the partial x-ray sources of the first group of the partial x-ray sources, and the second x-ray spectrum by applying a second acceleration voltage at the partial x-ray sources of the second group of the partial x-ray sources.

3. The computed tomography system of claim 2, wherein the annular x-ray source comprises a first voltage connection to generate an acceleration voltage for the first group of the partial x-ray sources and a second voltage connection to generate an acceleration voltage for the second group of the partial x-ray sources.

4. The computed tomography system of claim 1, wherein the computed tomography system is configured to activate at least one of the partial x-ray sources of the first group of the partial x-ray sources and at least one of the partial x-ray sources of the second group of the partial x-ray sources alternately.

5. The computed tomography system of claim 1, wherein the partial x-ray sources are on the annular x-ray source in such a way that a partial x-ray source of the first group and a partial x-ray source of the second group are arranged alternately in a circumferential direction.

6. The computed tomography system of claim 5, wherein the computed tomography system is configured to activate the partial x-ray sources in a sequence of arrangements of the partial x-ray sources, respectively, along a circular circumference.

7. The computed tomography system of claim 1, wherein the computed tomography system is configured to generate the second x-ray spectrum by adapting x-radiation generated by the second group of partial x-ray sources using x-ray filters.

8. The computed tomography system of claim 7, wherein the computed tomography system is configured to generate the first x-ray spectrum adapting x-radiation generated by the first group of partial x-ray sources using further x-ray filters,
and the further x-ray filters optionally differ from the x-ray filters of the second group in respect of their filter properties.

9. The computed tomography system of claim 1, wherein at least one of
the annular x-ray source comprises filter slots configured to receive x-ray filters, the filter slots at least in front of the second group of partial x-ray sources, or
the annular x-ray source comprises x-ray filters at least in front of the second group of partial x-ray sources.

10. The computed tomography system of claim 1, wherein the computed tomography system is configured to generate, during the computed tomography scan, one or more further x-ray spectra via one or more further groups of the partial x-ray sources.

11. The computed tomography system of claim 1, wherein the detector annulus comprises photon-counting detectors configured to distinguish between at least two energy thresholds.

12. The computed tomography system of claim 1, wherein the x-ray detectors are provided as detector pairs, a first x-ray detector of at least one detector pair is configured to absorb essentially low-energy x-radiation and a second x-ray detector of the at least one detector pair is configured to absorb essentially high-energy x-radiation.

13. The computed tomography system of claim 1, wherein the annular x-ray source and the detector annulus are partial annuli which are opposite each other such that the examination region is between the annular x-ray source and the detector annulus.

14. The computed tomography system of claim 1, wherein the annular x-ray source and the detector annulus are distributed over a complete annulus in each case.

15. A method for operating a computed tomography system which has a plurality of partial x-ray sources and a plurality of x-ray detectors, each being operated in a positionally fixed manner and surrounding an examination region, the method comprising:
activating a first partial x-ray source or a first subgroup of partial x-ray sources to generate first x-radiation directed at the examination region, the x-radiation having a first x-ray spectrum;
activating a second partial x-ray source or a second subgroup of partial x-ray sources to generate second x-radiation directed at the examination region having a second x-ray spectrum different than the first x-ray spectrum; and
detecting x-ray beams of the first x-ray spectrum and the second x-ray spectrum which pass through the examination region via the x-ray detectors.

16. The computed tomography system of claim 8, wherein the further x-ray filters differ from the x-ray filters of the second group with respect to filter properties.

17. The computed tomography system of claim 2, wherein the computed tomography system is configured to activate at least one of the partial x-ray sources of the first group of the partial x-ray sources and at least one of the partial x-ray sources of the second group of the partial x-ray sources alternately.

18. The computed tomography system of claim 17, wherein the partial x-ray sources are on the annular x-ray source in such a way that a partial x-ray source of the first group and a partial x-ray source of the second group are arranged alternately in a circumferential direction.

19. The computed tomography system of claim 18, wherein the computed tomography system is configured to activate the partial x-ray sources in a sequence of arrangements of the partial x-ray sources, respectively, on the circular circumference.

20. The method of claim 15, wherein the activating the first partial x-ray source or the first subgroup of partial x-ray sources and the activating the second partial x-ray source or the second subgroup of partial x-ray sources are performed in a time-staggered manner.
